(19) United States
(12) Patent Application Publication
(10) Pub. No.: US 2022/0101453 A1
(43) Pub. Date: Mar. 31, 2022

(54) METHOD, SYSTEM, AND COMPUTER PROGRAM PRODUCT FOR CREATING A RECIPE FOR A CHEMICAL COMPOSITION

(71) Applicant: Covestro LLC, Pittsburgh, PA (US)

(72) Inventor: David D. Steppan, Gibsonia, PA (US)

(21) Appl. No.: 17/425,381

(22) PCT Filed: Jan. 29, 2020
(86) PCT No.: PCT/US2020/015623

§ 371 Date: Jul. 23, 2021

Related U.S. Application Data
(60) Provisional application No. 62/799,340, filed on Jan. 31, 2019.

(51) Int. Cl. G06Q 50/04; G16C 20/10; G16C 20/30; G06Q 10/04

(52) U.S. Cl. CPC G06Q 50/04; G06Q 10/04; G16C 20/30; G16C 20/10

(57) ABSTRACT

A method of creating a recipe for a chemical composition, includes: receiving a property input from a user including at least one target physical property for a polymeric product to be produced from the chemical composition; receiving an initial chemical component for inclusion in the recipe; determining whether inclusion of the one or more initial chemical components in the recipe would produce the polymeric product having the at least one target physical property; generating, an advisory component recommendation comprising one or more additional or alternative chemical components for inclusion in the recipe; and modifying the recipe to include the one or more additional or alternative chemical components. A system and a computer program product for creating a recipe for a chemical composition are also disclosed.

### BACKGROUND

### Field

This disclosure relates to a method, system, and computer program product for creating a recipe for a chemical composition and, in a non-limiting embodiment, preparing the chemical composition used to produce a polymeric product having a target physical property.

### Description of Related Art

Developers of polymeric products commonly seek to produce a polymeric product having at least one target physical property (e.g., hardness, conductivity, Young's Modulus, etc.) within a predetermined range. For instance, the polymeric product may be required to exhibit a particular physical property within a predetermined range in order for that polymeric product to be suitable for a given end use.

Existing methods, systems, and computer program products for developing recipes for creating chemical compositions used to produce the polymeric product require a user to specify the components of the recipe based solely on that user's knowledge and/or intuition, without providing any guidance or recommendations regarding whether those components are suitable in combination for yielding the polymeric product having the desired physical property. In this way, the user does not know until the polymeric product has actually been created whether the desired physical property requirement has been satisfied. This known process can lead to wasted time and resources in the event that the polymeric product does not satisfy the desired physical property.

### SUMMARY

According to a non-limiting embodiment or aspect, provided is a method of creating a recipe for a chemical composition, including: receiving, via a user interface, a property input from a user, the property input including at least one target physical property for a polymeric product to be produced from the chemical composition; receiving, via a user interface, one or more initial chemical components for inclusion in the recipe for preparing the chemical composition; based on the at least one target physical property, determining, with at least one processor, whether inclusion of the one or more initial chemical components in the recipe would produce the polymeric product having the at least one target physical property; generating, with at least one processor, an advisory component recommendation including one or more additional or alternative chemical components for inclusion in the recipe; and in response to a user accepting the advisory component recommendation, modifying the recipe to include the one or more additional or alternative chemical components.

In one non-limiting embodiment or aspect, the method may further include: receiving, via a user interface, one or more subsequent chemical components for inclusion in the modified recipe; based on the at least one target physical property, determining, with at least one processor, whether inclusion of the one or more subsequent chemical components in the modified recipe would produce the polymeric product having the at least one target physical property; generating, with at least one processor, an updated advisory component recommendation including one or more additional or alternative chemical components; and in response to a user accepting the updated advisory component recommendation, modifying the modified recipe to form an updated recipe for preparing the chemical composition to include the one or more additional or alternative chemical components.

In one non-limiting embodiment or aspect, the method may further include storing the modified recipe in a saved recipes database. The method may further include programming or configuring a composition preparation device to prepare the chemical composition based on the modified recipe. The method may further include preparing, using the composition preparation device, the chemical composition based on the modified recipe. The method may further include: during preparation of the chemical composition, receiving, with at least one processor and from the composition preparation device, feedback data associated with a physical property of the chemical composition being prepared; and generating, with at least one processor, an adjustment component recommendation based on the feedback data. The one or more additional or alternative chemical components may include a reactive component and/or a non-reactive component.

In one non-limiting embodiment or aspect, determining whether inclusion of the one or more initial chemical components in the recipe would produce the polymeric product having the at least one target physical property may include: receiving, with at least one processor, chemical component data associated with at least one chemical component, the chemical component data stored in a chemical component database; and determining, based on the chemical component data and with at least one processor, whether the at least one chemical component stored in the chemical component database is suitable for inclusion in the recipe.

In one non-limiting embodiment or aspect, determining whether inclusion of the one or more initial chemical components in the recipe would produce the polymeric product having the at least one target physical property may include: receiving, with at least one processor, at least one of: (a) physical property data associated with a previously-prepared and tested chemical composition, the previously-prepared and tested chemical composition comprising at least one chemical component; or (b) physical property data from a predictive model generated for at least one chemical component; and determining, based on the physical property data and with at least one processor, whether the at least one chemical component is suitable for inclusion in the recipe. Receiving one or more initial chemical components for inclusion in the recipe may include receiving an amount for at least one of the one or more initial chemical components to be included in the recipe.

According to a non-limiting embodiment or aspect, provided is a method of preparing a chemical composition, including: receiving, via a user interface, a property input from a user, the property input including at least one target physical property for a polymeric product to be produced from the chemical composition; receiving, via a user interface, one or more initial chemical components for inclusion in the recipe for preparing the chemical composition; based on the at least one target physical property, determining, with at least one processor, whether inclusion of the one or more initial chemical components in the recipe would produce the polymeric product having the at least one target physical property; generating, with at least one processor, an advisory component recommendation including one or more additional or alternative chemical components for inclusion in the recipe; in response to a user accepting the advisory component recommendation, modifying the recipe to include the one or more additional or alternative chemical components; and preparing the chemical composition based on the modified recipe.

In one non-limiting embodiment or aspect, the method may include using a programmed or configured composition preparation device to prepare the chemical composition based on the modified recipe.

According to a non-limiting embodiment or aspect, provided is a computer program product for creating a recipe for a chemical composition including at least one non-transitory computer-readable medium including one or more instructions that, when executed by at least one processor, cause the at least one processor to: receive, via a user interface, a property input from a user, the property input including at least one target physical property for a polymeric product to be produced from the chemical composition; receive, via a user interface, one or more initial chemical components for inclusion in the recipe for preparing the chemical composition; based on the at least one target physical property, determine whether inclusion of the one or more initial chemical components in the recipe would produce the polymeric product having the at least one target physical property; generate an advisory component recommendation including one or more additional or alternative chemical components for inclusion in the recipe; and in response to a user accepting the advisory component recommendation, modify the recipe to include the one or more additional or alternative chemical components.

In one non-limiting embodiment or aspect, the one or more instructions may further cause the at least one processor to: in response to a user rejecting the advisory component recommendation, receive, via a user interface, one or more alternative or additional subsequent chemical components for inclusion in the recipe; based on the at least one target physical property, determine whether inclusion of the one or more subsequent alternative or additional chemical components in the recipe would produce the polymeric product having the at least one target physical property; generate an updated advisory component recommendation comprising one or more additional or alternative chemical components; in response to a user accepting the updated advisory component recommendation, modify the recipe to form an updated recipe for preparing the chemical composition to include the one or more additional or alternative chemical components. The one or more instructions may further cause the at least one processor to program or configure a composition preparation device to prepare the chemical composition based on the modified recipe. The one or more instructions may further cause the at least one processor to prepare, using the composition preparation device, the chemical composition based on the modified recipe. The one or more instructions may further cause the at least one processor to: during preparation of the chemical composition, receive from the composition preparation device, feedback data associated with a physical property of the chemical composition being prepared; and generate an adjustment component recommendation based on the feedback data.

According to a non-limiting embodiment or aspect, provided is a system for creating a recipe for a chemical composition including at least one processor programmed or configured to: receive, via a user interface, a property input from a user, the property input including at least one target physical property for a polymeric product to be produced from the chemical composition; receive, via a user interface, one or more initial chemical components for inclusion in the recipe for preparing the chemical composition; based on the at least one target physical property, determine whether inclusion of the one or more initial chemical components in the recipe would produce the polymeric product having the at least one target physical property; generate an advisory component recommendation including one or more additional or alternative chemical components for inclusion in the recipe; and in response to a user accepting the advisory component recommendation, modify the recipe to include the one or more additional or alternative chemical components.

In one non-limiting embodiment or aspect, the at least one processor may be further programmed or configured to: in response to a user rejecting the advisory component recommendation, receive, via a user interface, one or more alternative or additional subsequent chemical components for inclusion in the recipe; based on the at least one target physical property, determine whether inclusion of the one or more subsequent alternative or additional chemical components in the recipe would produce the polymeric product having the at least one target physical property; generate an updated advisory component recommendation comprising one or more additional or alternative chemical components; in response to a user accepting the updated advisory component recommendation, modify the recipe to form an updated recipe for preparing the chemical composition to include the one or more additional or alternative chemical components. The system may further include a composition preparation device, and the at least one processor may be further programmed or configured to program the composition preparation device to prepare the chemical composition based on the modified recipe. The composition preparation device may be configured to prepare the chemical composition based on the modified recipe. During preparation of the chemical composition, the composition preparation device may be configured to communicate feedback data to the at least one processor, the feedback data associated with a physical property of the chemical composition being prepared; and the at least one processor may be configured to generate an adjustment component recommendation based on the feedback data. The at least one processor may be further programmed or configured to store the modified recipe in a saved recipes database.

Further embodiments or aspects are set forth in the following numbered clauses:

Clause 1: A method of creating a recipe for a chemical composition, comprising: receiving, via a user interface, a property input from a user, the property input comprising at least one target physical property for a polymeric product to be produced from the chemical composition; receiving, via a user interface, one or more initial chemical components for inclusion in the recipe for preparing the chemical composition; based on the at least one target physical property, determining, with at least one processor, whether inclusion of the one or more initial chemical components in the recipe would produce the polymeric product having the at least one target physical property; generating, with at least one processor, an advisory component recommendation comprising one or more additional or alternative chemical components for inclusion in the recipe; and in response to a user accepting the advisory component recommendation, modifying the recipe to include the one or more additional or alternative chemical components.

Clause 2: The method of clause 1, further comprising: receiving, via a user interface, one or more subsequent chemical components for inclusion in the modified recipe; based on the at least one target physical property, determining, with at least one processor, whether inclusion of the one or more subsequent chemical components in the modified recipe would produce the polymeric product having the at least one target physical property; generating, with at least one processor, an updated advisory component recommendation comprising one or more additional or alternative chemical components; in response to a user accepting the updated advisory component recommendation, modifying the modified recipe to form an updated recipe for preparing the chemical composition to include the one or more additional or alternative chemical components.

Clause 3: The method of clause 1 or 2, further comprising: storing the modified recipe in a saved recipes database.

Clause 4: The method of any of clauses 1-3, further comprising: programming or configuring a composition preparation device to prepare the chemical composition based on the modified recipe.

Clause 5: The method of clause 4, further comprising: preparing, using the composition preparation device, the chemical composition based on the modified recipe.

Clause 6: The method of clause 5, further comprising: during preparation of the chemical composition, receiving, with at least one processor and from the composition preparation device, feedback data associated with a physical property of the chemical composition being prepared; and generating, with at least one processor, an adjustment component recommendation based on the feedback data.

Clause 7: The method of any of clauses 1-6, wherein the one or more additional or alternative chemical components comprises a reactive component and/or a non-reactive component.

Clause 8: The method of any of clauses 1-7, wherein determining whether inclusion of the one or more initial chemical components in the recipe would produce the polymeric product having the at least one target physical property comprises: receiving, with at least one processor, chemical component data associated with at least one chemical component, the chemical component data stored in a chemical component database; and determining, based on the chemical component data and with at least one processor, whether the at least one chemical component stored in the chemical component database is suitable for inclusion in the recipe.

Clause 9: The method of any of clauses 1-8, wherein determining whether inclusion of the one or more initial chemical components in the recipe would produce the polymeric product having the at least one target physical property comprises: receiving, with at least one processor, at least one of: (a) physical property data associated with a previously-prepared and tested chemical composition, the previously-prepared and tested chemical composition comprising at least one chemical component; or (b) physical property data from a predictive model generated for at least one chemical component; and determining, based on the physical property data and with at least one processor, whether the at least one chemical component is suitable for inclusion in the recipe.

Clause 10: The method of any of clauses 1-9, wherein receiving one or more initial chemical components for inclusion in the recipe comprises receiving an amount for at least one of the one or more initial chemical components to be included in the recipe.

Clause 11: A method of preparing a chemical composition, comprising: receiving, via a user interface, a property input from a user, the property input comprising at least one target physical property for a polymeric product to be produced from the chemical composition; receiving, via a user interface, one or more initial chemical components for inclusion in the recipe for preparing the chemical composition; based on the at least one target physical property, determining, with at least one processor, whether inclusion of the one or more initial chemical components in the recipe would produce the polymeric product having the at least one target physical property; generating, with at least one processor, an advisory component recommendation comprising one or more additional or alternative chemical components for inclusion in the recipe; in response to a user accepting the advisory component recommendation, modifying the recipe to include the one or more additional or alternative chemical components; and preparing the chemical composition based on the modified recipe.

Clause 12: The method of clause 11, comprising using a programmed or configured composition preparation device to prepare the chemical composition based on the modified recipe.

Clause 13: A computer program product for creating a recipe for a chemical composition comprising at least one non-transitory computer-readable medium including one or more instructions that, when executed by at least one processor, cause the at least one processor to: receive, via a user interface, a property input from a user, the property input comprising at least one target physical property for a polymeric product to be produced from the chemical composition; receive, via a user interface, one or more initial chemical components for inclusion in the recipe for preparing the chemical composition; based on the at least one target physical property, determine whether inclusion of the one or more initial chemical components in the recipe would produce the polymeric product having the at least one target physical property; generate an advisory component recommendation comprising one or more additional or alternative chemical components for inclusion in the recipe; and in response to a user accepting the advisory component recommendation, modify the recipe to include the one or more additional or alternative chemical components.

Clause 14: The computer program product of clause 13, wherein the one or more instructions further cause the at least one processor to: in response to a user rejecting the advisory component recommendation, receive, via a user interface, one or more alternative or additional subsequent chemical components for inclusion in the recipe; based on the at least one target physical property, determine whether inclusion of the one or more subsequent alternative or additional chemical components in the recipe would produce the polymeric product having the at least one target physical property; generate an updated advisory component recommendation comprising one or more additional or alternative chemical components; in response to a user accepting the updated advisory component recommendation, modify the recipe to form an updated recipe for preparing the chemical composition to include the one or more additional or alternative chemical components.

Clause 15: The computer program product of clause 13, wherein the one or more instructions further cause the at least one processor to: program or configure a composition preparation device to prepare the chemical composition based on the modified recipe.

Clause 16: The computer program product of clause 15, wherein the one or more instructions further cause the at least one processor to: prepare, using the composition preparation device, the chemical composition based on the modified recipe.

Clause 17: The computer program product of clause 15 or 16, wherein the one or more instructions further cause the at least one processor to: during preparation of the chemical composition, receive from the composition preparation device, feedback data associated with a physical property of the chemical composition being prepared; and generate an adjustment component recommendation based on the feedback data.

Clause 18: A system for creating a recipe for a chemical composition comprising at least one processor programmed or configured to: receive, via a user interface, a property input from a user, the property input comprising at least one target physical property for a polymeric product to be produced from the chemical composition; receive, via a user interface, one or more initial chemical components for inclusion in the recipe for preparing the chemical composition; based on the at least one target physical property, determine whether inclusion of the one or more initial chemical components in the recipe would produce the polymeric product having the at least one target physical property; generate an advisory component recommendation comprising one or more additional or alternative chemical components for inclusion in the recipe; and in response to a user accepting the advisory component recommendation, modify the recipe to include the one or more additional or alternative chemical components.

Clause 19: The system of clause 18, wherein the at least one processor is further programmed or configured to: in response to a user rejecting the advisory component recommendation, receive, via a user interface, one or more alternative or additional subsequent chemical components for inclusion in the recipe; based on the at least one target physical property, determine whether inclusion of the one or more subsequent alternative or additional chemical components in the recipe would produce the polymeric product having the at least one target physical property; generate an updated advisory component recommendation comprising one or more additional or alternative chemical components; in response to a user accepting the updated advisory component recommendation, modify the recipe to form an updated recipe for preparing the chemical composition to include the one or more additional or alternative chemical components.

Clause 20: The system of clause 18, further comprising a composition preparation device, wherein the at least one processor is further programmed or configured to: program or configure the composition preparation device to prepare the chemical composition based on the modified recipe.

Clause 21: The system of clause 20, wherein the composition preparation device is configured to prepare the chemical composition based on the modified recipe.

Clause 22: The system of clause 20 or 21, wherein during preparation of the chemical composition, the composition preparation device is configured to communicate feedback data to the at least one processor, the feedback data associated with a physical property of the chemical composition being prepared; and the at least one processor is configured to generate an adjustment component recommendation based on the feedback data.

Clause 23: The system of any of clauses 18-22, wherein the at least one processor is further programmed or configured to: store the modified recipe in a saved recipes database.

Clause 24: A method of creating a recipe for a chemical composition, comprising: receiving, via a user interface, a property input from a user, the property input comprising at least one target physical property for a polymeric product to be produced from the chemical composition; receiving, via a user interface, one or more initial chemical components for inclusion in the recipe for preparing the chemical composition; based on the at least one target physical property, determining, with at least one processor, whether inclusion of the one or more initial chemical components in the recipe would produce the polymeric product having the at least one target physical property; generating, with at least one processor, an advisory component recommendation comprising one or more additional or alternative chemical components for inclusion in the recipe; in response to a user rejecting the advisory component recommendation, receiving, via a user interface, one or more alternative or additional subsequent chemical components for inclusion in the recipe; based on the at least one target physical property, determining, with at least one processor, whether inclusion of the one or more subsequent alternative or additional chemical components in the recipe would produce the polymeric product having the at least one target physical property; generating, with at least one processor, an updated advisory component recommendation comprising one or more additional or alternative chemical components; in response to a user accepting the updated advisory component recommendation, modifying the recipe to form an updated recipe for preparing the chemical composition to include the one or more additional or alternative chemical components.

Clause 25: The method of clause 24, further comprising: preparing the chemical composition based on the updated recipe.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments that are illustrated in the accompanying schematic figures, in which:

FIG. 1 is a schematic view of a system for creating a recipe for a chemical composition;

FIG. 2 is a schematic view of a composition preparation device;

FIGS. 3-11 are various schematic views of user interfaces for creating a recipe for a chemical composition;

FIG. 12 is a step diagram of a method for creating a recipe for a chemical composition; and

FIG. 13 is a process flow diagram of a method for creating a recipe for a chemical composition.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects. Hence, specific dimensions and other physical characteristics related to the embodiments or aspects disclosed herein are not to be considered as limiting.

As used herein, the terms “communication” and “communicate” may refer to the reception, receipt, transmission, transfer, provision, and/or the like, of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet, and/or the like) that includes data. It will be appreciated that numerous other arrangements are possible.

As used herein, the term “computing device” may refer to one or more electronic devices that are configured to directly or indirectly communicate with or over one or more networks. The computing device may be a mobile device. As an example, a mobile device may include a cellular phone (e.g., a smartphone or standard cellular phone), a portable computer (e.g., laptop computer or tablet computer), a wearable device (e.g., watches, glasses, lenses), a personal digital assistant (PDA), and/or other like devices. In other non-limiting embodiments, the computing device may be a desktop computer or other non-mobile computer. Furthermore, the term “computer” may refer to any computing device that includes the necessary components to receive, process, and output data, and normally includes a display, a processor, a memory, an input device, and a network interface. An “interface” refers to a generated display, such as one or more graphical user interfaces (GUIs) with which a user may interact, either directly or indirectly (e.g., through a keyboard, mouse, etc.). Further, one or more computers, e.g., servers, or other computerized devices, directly or indirectly communicating in the network environment may constitute a “system”.

Non-limiting embodiments or aspects are directed to a method, system, and computer program product for creating a recipe for a chemical composition. Non-limiting embodiments allow for a user to specify a target physical property of a polymeric product to be produced from the chemical composition and one or more initial components for inclusion in the recipe. Non-limiting embodiments allow for a recommendation system to determine, based on the target physical property, whether the chemical components selected by the user are suitable for inclusion in the recipe (whether the recipe would yield a polymeric product having the at least one target physical property). The recommendation system may generate an advisory component recommendation that suggests additional or alternative chemical components for inclusion in the recipe, in order to attain the polymeric product having the target physical property. Non-limiting embodiments provide for a back-and-forth interaction between the user and the recommendation system, such that the user can review, accept, or reject the advisory component recommendations of the recommendation system. This interactive communication between the user and the recommendation system may include an iterative process in which the user and recommendation system communicate with one another until the recipe would generate a polymeric product having the target physical property. Thus, the user may maintain control over the recipe, while still receiving recommendations from the system. Non-limiting embodiments allow for the user to receive recommendations for the recipe and to know, before any components are physically mixed together, whether the recipe would yield the target physical property.

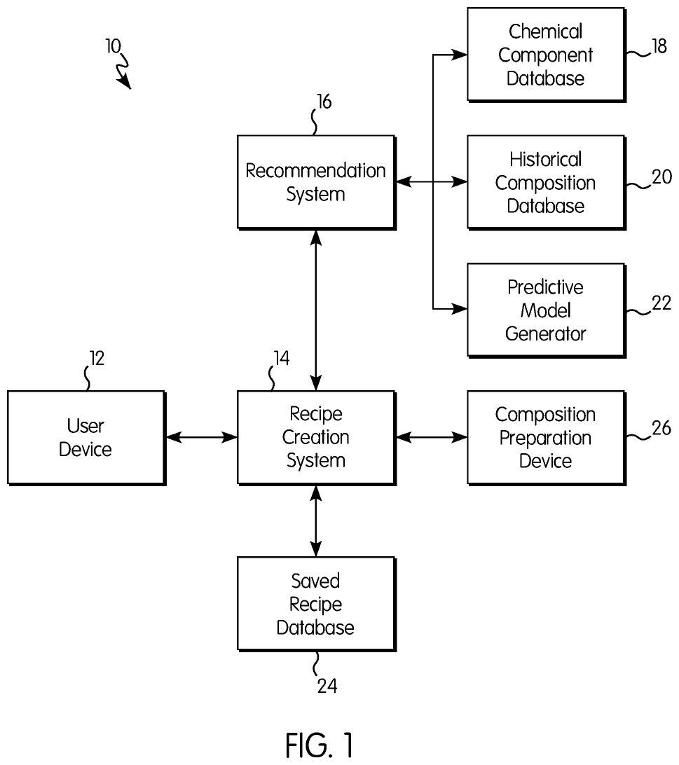

Referring to FIG. 1, a system 10 for creating a recipe for a chemical composition is shown. The system 10 may include a user device 12 in communication with a recipe creation system 14 in order to create a recipe for a chemical composition used to produce a polymeric product. The user device 12 may be a computing device and may include a screen to display at least one user interface (described hereinafter) for the user to interact with the RCS 14 to create the recipe for the chemical composition. The user may input into the user device 12 a property input specifying a target physical property or properties associated with a polymeric product to be produced from the chemical composition. The user may also input into the user device 12 an initial chemical component(s) for inclusion in the recipe. The specification of the initial chemical component(s) by the user may include the user specifying amounts of each component for inclusion in the recipe.

As used herein, the terms “polymer property” and “polymer properties” may refer to % Ash, % Intumescence, % Shrink, Compression Force Deflection (CFD), Indentation Force Deflection (IFD), IFD Recovery, Asker C Hardness, ASTM D 523 Specular Gloss at 20 and 60 Degrees, Ball Rebound, Burn Rate, measured in inches/minute, mm/minute or seconds, Coefficient of Linear Thermal Expansion (CLTE), Coefficient of Friction, Compression Set, may be measured as %, Compression Strength, Compression Strength at Deflection, Compression Load, Compression Recovery, Compressive Creep Properties, Density, Dielectric Strength, V/Mil, DIN 5377 CFD Tests, Drop Town Impact, Dynamic Modulus, Flexural Modulus, Flexural Properties, Gloss, Water Absorption, Humid Aged Compression Set (HACS), Haze Transmittance, Head and Neck Impact, Heat Sag, Internal Bond Strength, Melt Flow Rate, Melt Volume Rate, Melt Flow Stability, Melt Viscosity, may be measured in Pa-Sm Modulus of Elasticity, Moisture Content, Mold Shrinkage, Burn Resistance, measured by Federal Motor Vehicle Safety Standard No. 302, Percentage or Number of Open or Closed Cells, Percent Oxygen Index, Peel 90 Degrees, Peel 180 Degrees, Plunger Rebound, Puncture Test, Resiliency, Rigid Shear, Rockwell Hardness, Taber Abrasion, Scratch Hardness, Shear Strength, Shear Force, Shore A Hardness, Shore C Hardness, Shore D Hardness, Shore OO Hardness, Sled Dynamic Impact, Specific Gravity, Gloss, Distinctness of Image (DOI), Split Tear Strength, Specific Gravity, Specific Density, Tear Resistance, Tear Strength, Tensile Adhesion, Tensile Modulus, Tensile Strength, Tensile Adhesion Strength, Tensile Energy at Break, Water Absorption, Permeability, Yellowness Index, Thermal Conductivity and Chemical Resistance.

As used herein, the term “recipe” refers to the set of instructions used to prepare the chemical composition used to produce the polymeric product. The recipe may include the various chemical components included in the chemical composition. The recipe may include amounts of the chemical components included in the chemical composition. The recipe may also include processing steps for preparing the chemical composition, such as a temperature at which the reaction mixture is to be held, the length of time to mix the chemical components, the speed at which to run the mixer, the order at which to add the ingredients, and other like processing steps.

As used herein, chemical components refer to a chemical species to be included in the chemical composition, which chemical composition produces the polymeric product. The chemical components may include reactive or non-reactive components. The chemical components may include monomers and/or other components that react to form the polymeric product. The chemical components may include a catalyst used to accelerate the rate of the chemical reaction to form the polymeric product, without being consumed in the reaction. The chemical components may include other non-reactive additives, such as colorants, reaction retarders, cell regulators, emulsifiers, foam stabilizers, surfactants, UV and IR light stabilizers, flame retardants, hydrolysis and degradation stabilizers, anti-statics, release agents, impact modifiers, fillers, blowing agents and the like.

The RCS 14 may communicate with a recommendation system 16 in order to generate an advisory component recommendation(s) for the user regarding additional and/or alternative chemical components for inclusion in the recipe. The RCS 14 and the recommendation system 16 may be separate systems or may be parts of the same system. The RCS 14 and/or the recommendation system 16 may determine whether inclusion of the chemical components input by the user would produce the polymeric product having the specified target physical property.

To determine whether inclusion of the chemical components input by the user would produce the polymeric product having the specified target physical property, the recommendation system may communicate with one or more of a chemical component database 18, a historical composition database 20, and a predictive model generator 22. Based on data from one or more of the chemical component database 18, the historical model database 22, or the predictive model generator 22, the recommendation system 16 may generate an advisory component recommendation to be communicated to the user (e.g., displayed on a user interface of the user device 12).

The chemical component database 18 may include chemical component data associated with at least one chemical component or combination of chemical components. This chemical component data may include property data associated with a particular chemical component or combination of chemical components. For example, this data may include property data found on a material safety data sheet (MSDS) or a technical data sheet. The chemical component data may include data associated with a manufacturer of the chemical component and/or the price associated with purchasing the chemical component. The chemical component data may include the chemical structure of the chemical component. The chemical component data may include data associated with the reactivity of that chemical component with other chemical components. The chemical component data may include data associated with properties commonly imparted by the chemical component in compositions or common uses of the chemical component in industry.

The historical composition database 20 may include physical property data associated with a previously-prepared and tested chemical composition. In this way, the recommendation system 16 may analyze and consider physical property data of similar known compositions to generate the advisory component recommendation for the user.

The predictive model generator 22 may generate a predictive model associated with physical property data for at least one chemical component. The predictive models generated by the predictive model generator 22 may incorporate physical property information of individual components or a portion of or the entire combination of all components and amounts used. The predictive model generator 22 may consider: physical property data of polymer compounds and individual components, amounts/recipes, and processing conditions. The predictive models may be generated using interpolations of existing data, database lookups of matches, multiple regression models of compositional effects on polymeric properties, or any number of machine learning and neural network algorithms. The predictive model generator 22 may generate: predictions of performance properties, recipes, and processing conditions.

Based at least in part on data from one or more of the chemical component database 18, the historical model database 22, or the predictive model generator 22, the recommendation system 16 may generate the advisory component recommendation including one or more additional or alternative chemical components for inclusion in the recipe. The advisory component recommendation may recommend that a chemical component(s) presently in the recipe be removed. The advisory component recommendation may recommend that an additional chemical component be added to the recipe. The advisory component recommendation may recommend that an alternative chemical component be substituted into the recipe for a chemical component already in the recipe. The advisory component recommendation may recommend that the amount of a chemical component already in the recipe be altered (increased or decreased). The advisory component recommendation may recommend processing steps for combining the chemical components. The advisory component recommendations may be displayed for the user on the display of the user device 12. The advisory component recommendation may include multiple alternative options for the user (see e.g., FIGS. 7, 8, and 10).

Upon the advisory component recommendation being displayed, the user may accept, reject, or modify the advisory component recommendation. The user may reject or modify the advisory component recommendation for any reason, such as the recommended chemical component or amount being unavailable, the recommended chemical component or amount being too costly, the recommended chemical component or amount being unsuitable to satisfy a physical property not previously specified to the system 10, or any other reason.

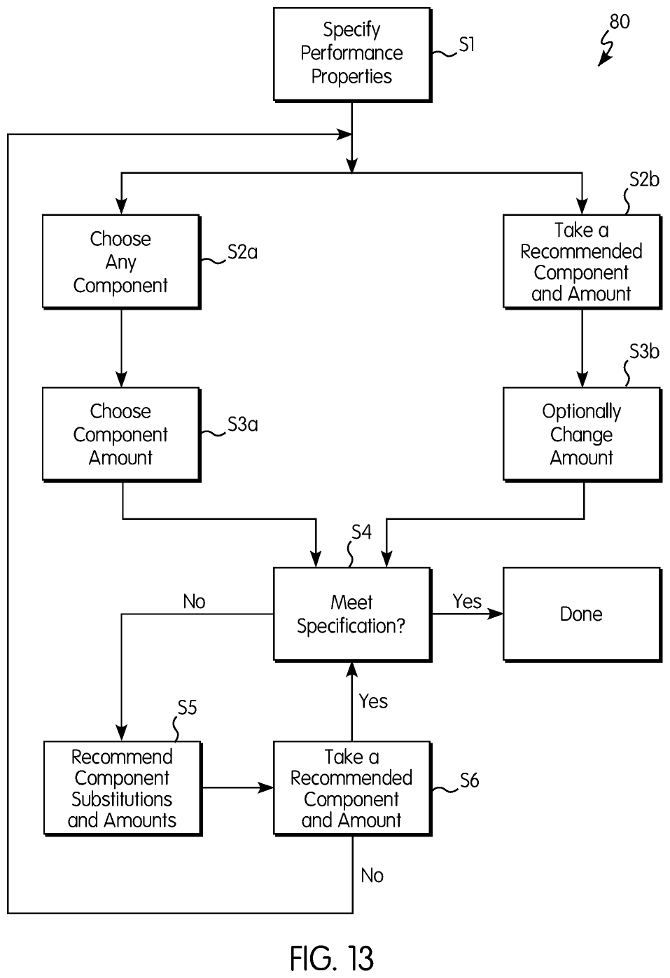

In response to the user rejecting and/or modifying the advisory component recommendation, the user may specify one or more alternative or additional subsequent chemical components for inclusion in the recipe. Based on the at least one target physical property, the recommendation system 16 may determine, whether inclusion of the one or more subsequent alternative or additional chemical components in the recipe would produce the polymeric product having the at least one target physical property and generate an updated advisory component recommendation comprising one or more additional or alternative chemical components. The user may accept or reject the updated advisory component recommendation. The user and the recommendation system 16 may continue this iterative and interactive process of the user specifying additional or alternative chemical components for inclusion in the recipe and the recommendation system 16 determining whether the recipe would produce the polymeric product having the at least one target physical property and generating an updated advisory component recommendation comprising one or more additional or alternative chemical components until the user is satisfied with the recipe and the recipe is determined to produce the polymeric product having the at least one target physical property (see FIG. 13 for a non-limiting example of the iterative and interactive process flow).

In response to the user accepting the advisory component recommendation, the RCS 14 may modify the recipe to include the recommended additional or alternative chemical components.

In addition to the initial chemical components input by the user and the advisory component recommendation, the user device 12 and the RCS 14 may communicate back-and-forth in an iterative process until the user is satisfied with the recipe or modified recipe (hereinafter the “recipe”). In some non-limiting examples, the user may input subsequent chemical component(s) for inclusion of the recipe into the user device 12, which subsequent chemical component may be communicated to the RCS 14. Based on the target physical property, the recommendation system 16 may determine whether the subsequent chemical components are suitable for inclusion in the recipe to produce the polymeric product having the target physical property (as previously described). The recommendation system 16 may further generate an updated advisory component recommendation including one or more additional or alternative chemical components. The user may accept, reject, or modify the updated advisory component recommendation as described above. Upon the user accepting the updated advisory component recommendation, the RCS 14 may modify the recipe to form an updated recipe which includes the one or more additional or alternative chemical components.

Once the recipe is determined suitable for producing the polymeric product having the target physical property, the RCS 14 may communicate the recipe to a saved recipes database 24. The saved recipes database 24 may store recipes developed by the system, along with data associated with the target physical properties for which the polymeric product resulting from those recipes are suitable.

Once the recipe is determined suitable for producing the polymeric product having the target physical property, the RCS 14 may program or configure a composition preparation device 26 to prepare the chemical composition based on the recipe. Programming or configuring the composition preparation device may include communicating the recipe to the composition preparation device 26 and/or otherwise preparing the composition preparation device 26 such that the composition preparation device 26 is configured to prepare the chemical composition. The composition preparation device 26 may prepare the chemical composition based on the recipe.

In some non-limiting embodiments, during or after preparation of the chemical composition, the composition preparation device 26 may communicate feedback data to the RCS 14. The feedback may include ambient conditions, such as temperature and pressure, processing conditions, including changes in temperature and pressure, and data associated with a physical property of the chemical composition being prepared. Exemplary physical properties include conductivity, indentation force deflection, hardness shore A, hardness shore D, hysteresis, exotherm, torque, mass flow and other non-destructive tests. The RCS 14 and/or the recommendation system 16 may, in response, generate an adjustment component recommendation based on the feedback data. The adjustment component recommendation may include an additional or alternative chemical component (or amount thereof) or further processing instructions in order to modify the chemical composition attain the polymeric product having the target physical property. The user may accept, reject, or modify the adjustment component recommendation. The RCS 14 may then communicate the accepted or modified adjustment component recommendation to the composition preparation device 26 to produce the chemical composition. This process allows for in-preparation adjustments to be made to the chemical composition so that a suitable polymeric product may be achieved.

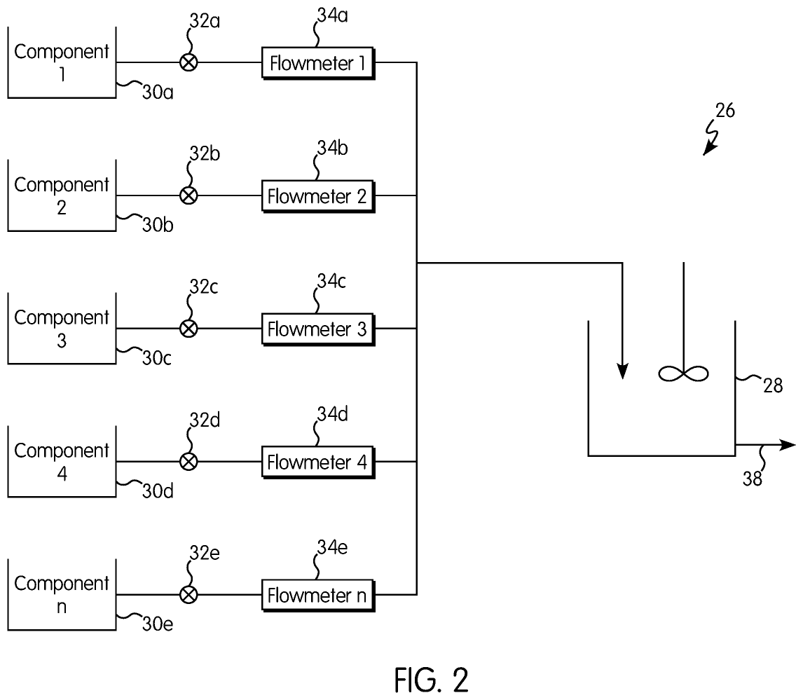

Referring to FIG. 2, a non-limiting example of the composition preparation device 26 is shown. The component preparation device 26 may include a mixer 28 for receiving and mixing the chemical components of the chemical composition. The component preparation device 26 may also include at least one component vat 30a-e, each component vat 30a-e containing at least one chemical component, such as any of the chemical components mentioned above. Each component vat 30a-e may be in fluid communication with the mixer 28 via a system of pipes for conveying the chemical components. A pump 32a-e may be provided to convey the chemical components from the component vats 30a-e to the mixer 28. Further, a flowmeter 34a-e may be provided to measure the amount of each chemical component flowing from the component vats 30a-e to the mixer 28. The flowmeter 34a-e may communicate with the corresponding pump 32a-e to control the pump speed. The chemical components may enter the mixer 28 via a component inlet 36. Upon mixing the chemical components of the chemical composition per the recipe, the resulting polymeric product may be flowed from the mixer 28 through a polymeric product outlet 38.

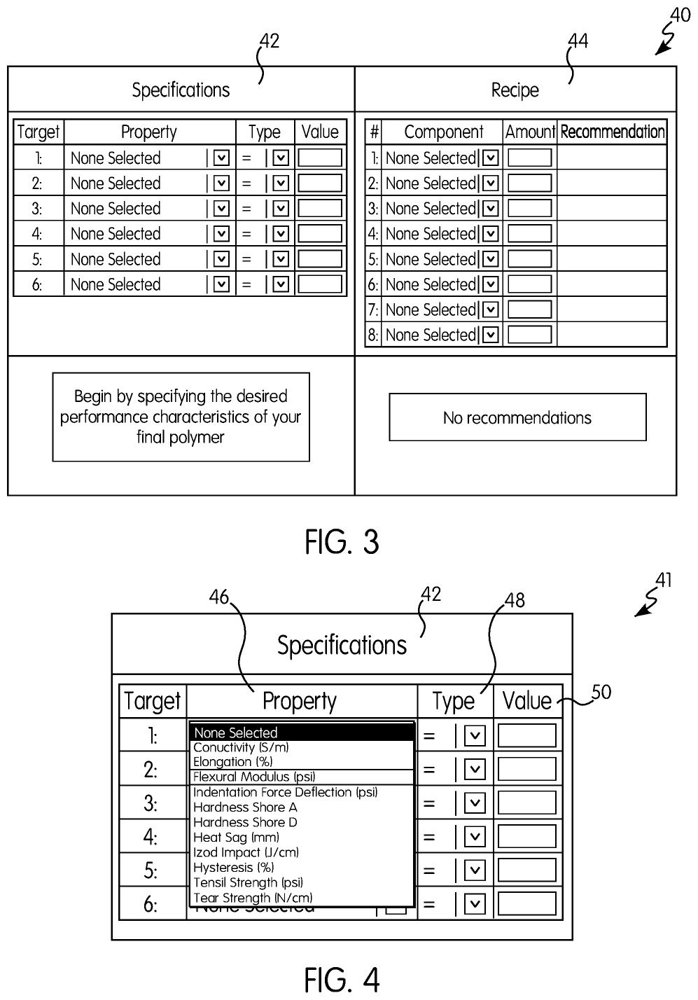

Referring to FIGS. 3-11, various non-limiting user interfaces displayed on the user device 12 are shown for the system, method, and computer program product for creating a recipe for a chemical composition. It will be appreciated that other arrangements of the user interface may be used for creating the recipe of the chemical composition.

Referring to FIG. 3, a first user interface 40 is shown. This first user interface 40 may be displayed at the start of the user creating a recipe for a chemical composition using the system described herein. The first user interface 40 may include a specification panel 42 and a recipe panel 44. The specification panel 42 may cover a portion of the first user interface 40 and allow the user to specify the target physical property data associated for the polymeric product to be produced from the chemical composition. The recipe panel 44 may cover a portion of the first user interface 40 and allow the user to specify the initial chemical components for inclusion in the recipe of the chemical composition. The specification panel 42 and the recipe panel 44 may include various fields for receiving data inputs from the user (e.g., target physical property data and/or initial chemical components). The various fields may include dropdown boxes, freeform fields, radio buttons, or any type of field commonly used in user interfaces.

Referring to FIG. 4, a second user interface 41 is shown. The second user interface 41 may include the specification panel 42, and FIG. 4 shows a non-limiting example of the specification panel 42 in more detail. The specification panel 42 may include a physical property option 46, a type option 48, and a value option 50. The physical property option 46 may allow the user to select the target physical property of the polymeric product. The user may select multiple target physical properties to be specified for the polymeric product. As shown in FIG. 4, the physical property option 46 may include a dropdown with a list of possible target physical properties to be selected. Non-limiting examples of target physical properties include conductivity, elongation, indentation force deflection, hardness shore A, hardness shore D, heat sag, Izod impact, hysteresis, tensile strength, tear strength, and any of the physical properties listed herein.

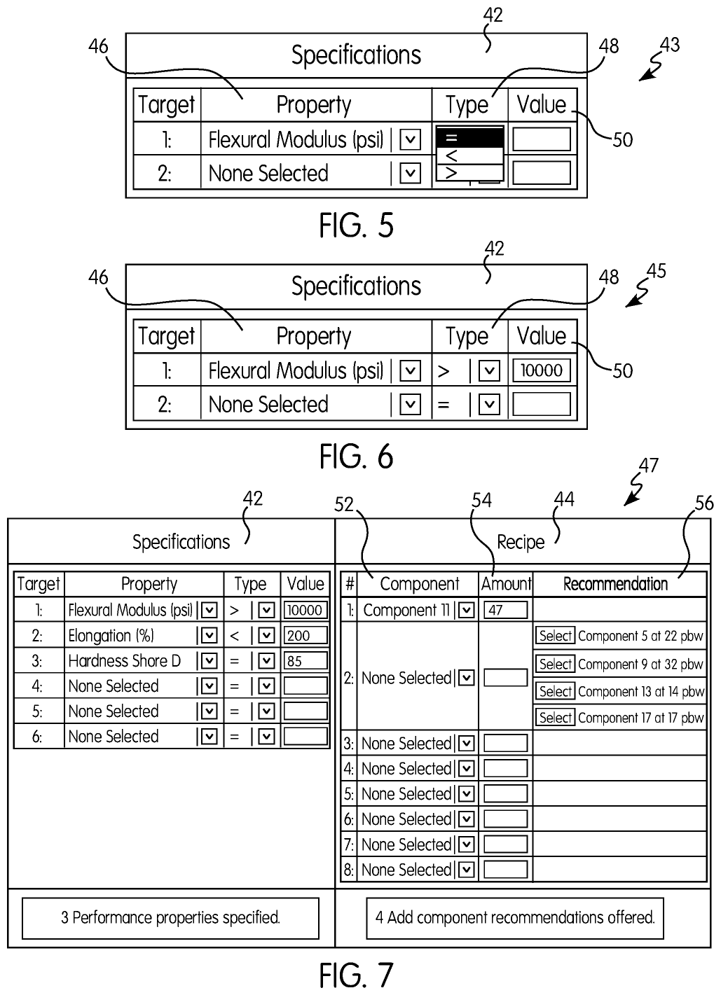

Referring to FIG. 5, a third user interface 43 is shown. The third user interface 43 may include the specification panel 42, and FIG. 5 shows a non-limiting example of the specification panel 42 in more detail and, specifically, the type option 48 of the specification panel 42. Upon selecting the target physical property using the physical property option 46, the user may select the type using the type option 48, the type option allowing the user to configure the range associated with the target physical property. As shown in FIG. 5, the type option 48 may include a dropdown with a list of possible range types to be selected. Non-limiting range options include greater than, less than, greater than or equal to, less than or equal to, equal to, and the like. A type option 48 may be selected for each specified physical property.

Referring to FIG. 6, a fourth user interface 45 is shown. The fourth user interface 45 may include the specification panel 42, and FIG. 6 shows a non-limiting example of the specification panel 42 in more detail and, specifically, the value option 50 of the specification panel 42. Upon selecting the target physical property using the physical property option 46 and the range type using the type option 48, the user may select the value of the range. As shown in FIG. 6, the value option 50 may be a freeform field configured for the user to enter a numerical and/or alphabetical value to be associated with the specified physical property. Thus, based on the options selected by the user in FIG. 6, the user is creating a recipe for a polymeric product which polymeric product has a flexural modulus greater than 10,000 psi.

Referring to FIG. 7, a fifth user interface 47 is shown. The fifth user interface 47 may include the specification panel 42 and the recipe panel 44. In FIG. 7, the user has specified three target physical properties for the polymeric product to be produced from the chemical composition (flexural modulus, elongation, and hardness shore D). The recipe panel 44 may include a recipe component option 52, a component amount option 54, and a component recommendation option 56. The recipe component option 52 may allow the user to specify at least one initial or subsequent chemical component for inclusion in the recipe. As shown in FIG. 7, the recipe component option 52 may be a dropdown with a list of possible chemical components for inclusion in the recipe. In this particular example, the user selects Component 11, which corresponds to a medium chain length polyether polyol having a hydroxyl value of 340 mg KOH/gram and a nominal functionality of 4.0. The component amount option 54 may allow the user to specify an amount of the specified chemical component. As shown in FIG. 7, the component amount option 54 may be a freeform field configured to receive a numerical entry by the user corresponding to the amount of the specified chemical component. In this particular example, the user specifies that the recipe includes 47 parts by weight (pbw) of Component 11.

With continued reference to FIG. 7, upon the user specifying an initial chemical component and corresponding amount thereof, the recommendation system immediately and automatically generates at least one recommendation, which is displayed in the component recommendation option 56 section of the recipe panel 44. The recommendation may include an additional and/or alternative chemical component for inclusion in the recipe, as previously described. In this particular example, the recommendation system recommends four potential second chemical components (Chemical Component 5, 9, 13, or 17) based on the user specifying Component 11 at 47 pbw. These recommended chemical components correspond to suitable chain extenders for the selected polyol. The recommendation also include a recommended amount (e.g., in pbw) for each of the recommended chemical components (e.g., include Component 5 at 22 pbw in the recipe). The component recommendation option may include a user selectable option, which allows the user to accept, reject, or modify the various generated recommendations. This option is shown in the non-limiting form of a “Select” button in FIG. 7.

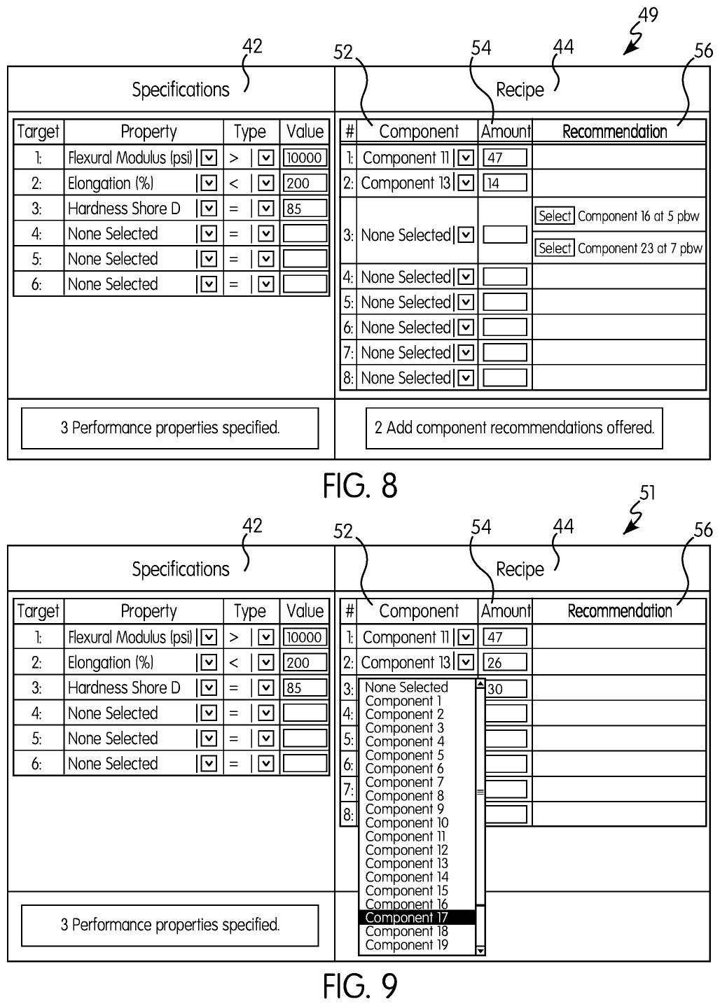

Referring to FIG. 8, a sixth user interface 49 is shown. The sixth user interface 49 includes the features shown and described in connection with the fifth user interface 47, except the user has selected one of the chain extender recommendations generated by the recommendation system. From the recommendations displayed in FIG. 7, the user selected the recommendation of Component 13 at 14 pbw. In response to the user selecting this recommendation, Component 13 is added to the recipe component option 52 and 14 pbw is added to the component amount option 54. In response to this second chemical component being added to the recipe, the recommendation system may automatically generate a subsequent recommendation to add an additional chemical component. In this example, the recommendation system recommends adding either Component 16 (a polyether polyol of functionality 2.2 and OH# of 35) at 16 pbw or Component 23 (a polyether polyol of functionality 2.0 and OH# of 30) at 7 pbw. It will be appreciated that the recommendation system may continue to iteratively recommend chemical components as the recipe is updated by adding or removing chemical components or altering their amounts.

Referring to FIG. 9, a seventh user interface 51 is shown. The seventh user interface 51 includes the features shown and described in connection with the sixth user interface 49, except as follows. As shown in FIG. 9, the user rejected the recommendation system's suggestion of Components 16 and 23. Instead, the user added Component 17 to the recipe at 30 pbw and updated the amount of Component 13 to 26 pbw. Component 17 is a modified methylene diphenyl diisocyanate (MDI) prepolymer at 22 NCO %. The system advantageously allows the user to dynamically interact with the recommendation system to customize the recipe by accepting or rejecting the recommendations and dynamically altering the recipe and having recommendations generated in response.

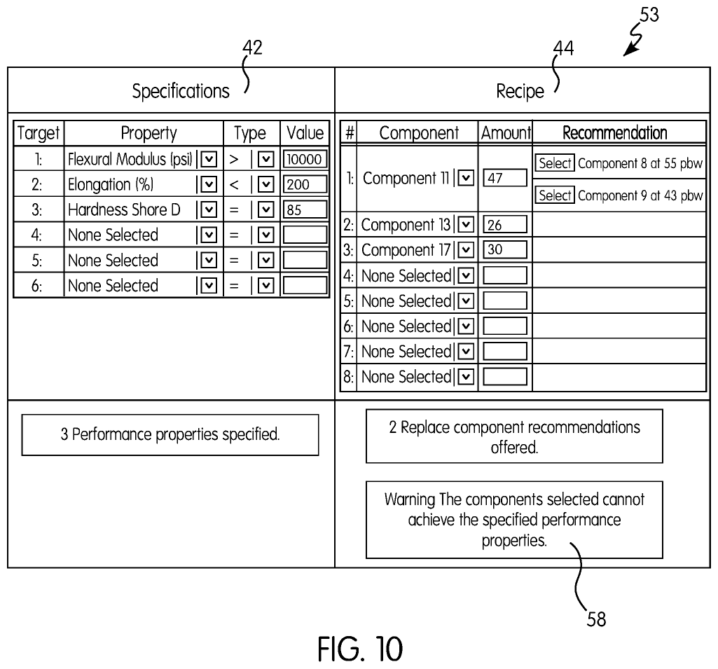

Referring to FIG. 10, an eighth user interface 53 is shown. The eighth user interface 53 includes the features shown and described in connection with the seventh user interface 51, except as follows. The recipe shown in the eighth user interface 53 is the same as the recipe selected by the user in the seventh user interface 51. In response to the user specifying the particular recipe, the recommendation system displays a system determination 58, which alerts the user as to whether the polymeric product formed from the recipe for the chemical composition would meet the target physical properties or not. In the non-limiting example shown in FIG. 10, the system determination 58 indicates that the selected chemical components and/or amounts could not achieve the target physical properties for the polymeric product. This result is because the hardness of the polymeric product would be in excess of the specified shore D hardness in the shown example. The reasons for the polymeric product not attaining the target physical properties may be displayed to the user. The recommendation system may display further recommendations in the component recommendation option 56 section, which it determines would modify the recipe so that the recipe for the chemical composition would produce the polymeric product having the target physical properties. In this particular non-limiting example, the recommendation system recommends replacing Component 11 with either Component 8 at 55 pbw or Component 9 at 43 pbw. Component 8 is a polyether polyol having a hydroxyl value of 28 with a nominal functionality of 2.0. Component 9 is another longer chain, a linear polyether polyol. The user may accept or reject the further recommendations.

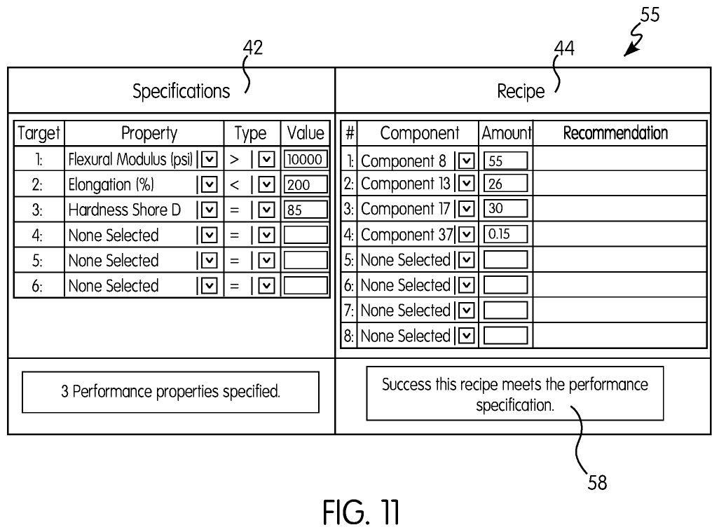

Referring to FIG. 11, a ninth user interface 55 is shown. The ninth user interface 55 includes the features shown and described in connection with the eighth user interface 53, except as follows. As shown in FIG. 11, the user accepted the recommendation system's suggestion of replacing Component 11 with Component 8. Furthermore, the system recommended the addition of a catalyst, which the user accepted, and added Component 37 to the recipe at 0.15 pbw. In response to these changes to the recipe by the user, the recommendation system displays a system determination 58 that notifies the user that the recipe meets the target physical properties. Thus, the recipe for the chemical composition shown in FIG. 11 would produce a polymeric product that meets the target physical properties from the specification panel 42.

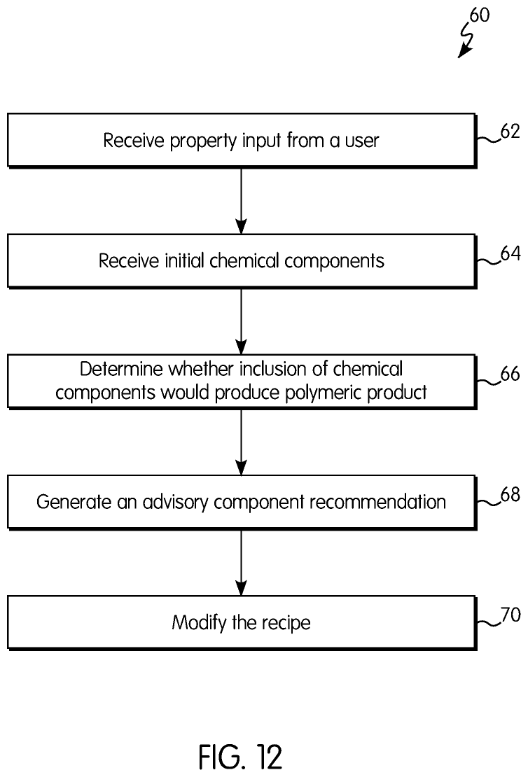

Referring to FIG. 12, a method 60 of creating a recipe for the chemical composition is shown. At a first step 62, the RCS 14 and/or the recommendation system 16 may receive, via a user input to a user interface displayed on the user device 12, the property input from a user, the property input comprising the at least one target physical property for a polymeric product to be produced from the chemical composition. At a second step 64, the RCS 14 and/or the recommendation system 16 may receive, via a user input to a user interface displayed on the user device 12, the one or more initial chemical components for inclusion in the recipe for preparing the chemical composition. At a third step 66, the RCS 14 and/or the recommendation system 16 may determine whether inclusion of the one or more initial chemical components in the recipe would produce the polymeric product having the at least one target physical property based on the target physical property input by the user. At a fourth step 68, the RCS 14 and/or the recommendation system 16 may generate an advisory component recommendation comprising one or more additional or alternative chemical components for inclusion in the recipe. At a fifth step 70, the RCS 14 and/or the recommendation system 16 may, in response to a user accepting the advisory component recommendation, modify the recipe to include the one or more additional or alternative chemical components. The composition preparation device 26 may prepare the chemical composition based on the modified recipe received from the RCS 14 and/or the recommendation system 16.

Referring to FIG. 13, a method 80 of creating a recipe for the chemical composition is shown. At a first step S1, the RCS 14 and/or the recommendation system 16 may receive, via a user input to a user interface displayed on the user device 12, the property input from a user, the property input comprising the at least one target physical property for a polymeric product to be produced from the chemical composition.

With continued reference to FIG. 13, at a second step S2a the RCS 14 and/or the recommendation system 16 may receive, via a user input to a user interface displayed on the user device 12, the one or more initial chemical components for inclusion in the recipe for preparing the chemical composition. At a third step S3a the RCS 14 and/or the recommendation system 16 may receive, via a user input to a user interface displayed on the user device 12, an amount(s) associated with the one or more initial chemical components for inclusion in the recipe for preparing the chemical composition.

With continued reference to FIG. 13, alternatively, at a second step S2b, the RCS 14 and/or the recommendation system 16 may recommend one or more initial chemical components and amounts thereof for inclusion in the recipe for preparing the chemical composition. At a third step S3b, the user may optionally change the amount of the recommended one or more initial chemical components.

At a fourth step S4, the RCS 14 and/or the recommendation system 16 may determine whether inclusion of the one or more initial chemical components in the recipe would produce the polymeric product having the at least one target physical property based on the target physical property input by the user. If the RCS 14 and/or the recommendation system 16 determine that the recipe would produce the polymeric product having the desired specifications, the method for creating a recipe may move to completion, and the composition preparation device 26 may prepare the chemical composition based on the recipe received from the RCS 14 and/or the recommendation system 16. Otherwise, the method may continue to a fifth step S5. At the fifth step S5, the RCS 14 and/or the recommendation system 16 may generate an advisory component recommendation comprising one or more additional or alternative chemical components for inclusion in the recipe. At a sixth step S6, the user may determine whether to accept or reject the advisory component recommendation. If the user decides to accept the advisory component recommendation, RCS 14 and/or the recommendation system 16 may, in response to a user accepting the advisory component recommendation, modify the recipe to include the one or more additional or alternative chemical components and proceed back to the fourth step S4 to confirm that the modified recipe would produce the polymeric product having the desired specifications. The composition preparation device 26 may then prepare the chemical composition based on the modified recipe received from the RCS 14 and/or the recommendation system 16. However, if the user decides to reject the advisory component recommendation, the method may proceed back to the second step S2a or S2b, during which the user may modify the recipe (e.g., components and/or amounts thereof). In this way, the method 80 of creating a recipe for the chemical composition may be an iterative process and an interactive process between the user and the RCS 14 and/or the recommendation system 16.

In a further non-limiting embodiment, a computer program product for creating a recipe for a chemical composition includes at least one non-transitory computer readable medium including program instructions that, when executed by at least one processor, cause the at least one processor to execute any of the systems and methods described herein. The at least one processor may include the RCS 14 and/or the recommendation system 16.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of creating a recipe for a chemical composition, comprising:
receiving, via a user interface, a property input from a user, the property input comprising at least one target physical property for a polymeric product to be produced from the chemical composition;
receiving, via a user interface, one or more initial chemical components for inclusion in the recipe for preparing the chemical composition;
based on the at least one target physical property, determining, with at least one processor, whether inclusion of the one or more initial chemical components in the recipe would produce the polymeric product having the at least one target physical property;
generating, with at least one processor, an advisory component recommendation comprising one or more additional or alternative chemical components for inclusion in the recipe; and
in response to a user accepting the advisory component recommendation, modifying the recipe to include the one or more additional or alternative chemical components.

2. The method of claim 1, further comprising:
receiving, via a user interface, one or more subsequent chemical components for inclusion in the modified recipe;
based on the at least one target physical property, determining, with at least one processor, whether inclusion of the one or more subsequent chemical components in the modified recipe would produce the polymeric product having the at least one target physical property;
generating, with at least one processor, an updated advisory component recommendation comprising one or more additional or alternative chemical components;
in response to a user accepting the updated advisory component recommendation, modifying the modified recipe to form an updated recipe for preparing the chemical composition to include the one or more additional or alternative chemical components.

3. The method of claim 1, further comprising:
storing the modified recipe in a saved recipes database.

4. The method of claim 1, further comprising:
programming or configuring a composition preparation device to prepare the chemical composition based on the modified recipe.

5. The method of claim 4, further comprising:
preparing, using the composition preparation device, the chemical composition based on the modified recipe.

6. The method of claim 5, further comprising:
during preparation of the chemical composition, receiving, with at least one processor and from the composition preparation device, feedback data associated with a physical property of the chemical composition being prepared; and
generating, with at least one processor, an adjustment component recommendation based on the feedback data.

7. The method of claim 1, wherein the one or more additional or alternative chemical components comprises a reactive component and/or a non-reactive component.

8. The method of claim 1, wherein determining whether inclusion of the one or more initial chemical components in the recipe would produce the polymeric product having the at least one target physical property comprises:
receiving, with at least one processor, chemical component data associated with at least one chemical component, the chemical component data stored in a chemical component database; and
determining, based on the chemical component data and with at least one processor, whether the at least one chemical component stored in the chemical component database is suitable for inclusion in the recipe.

9. The method of claim 1, wherein determining whether inclusion of the one or more initial chemical components in the recipe would produce the polymeric product having the at least one target physical property comprises:
receiving, with at least one processor, at least one of: (a) physical property data associated with a previously-prepared and tested chemical composition, the previously-prepared and tested chemical composition comprising at least one chemical component; or (b) physical property data from a predictive model generated for at least one chemical component; and determining, based on the physical property data and with at least one processor, whether the at least one chemical component is suitable for inclusion in the recipe.

10. The method of claim 1, wherein receiving one or more initial chemical components for inclusion in the recipe comprises receiving an amount for at least one of the one or more initial chemical components to be included in the recipe.

11. A method of preparing a chemical composition, comprising:
receiving, via a user interface, a property input from a user, the property input comprising at least one target physical property for a polymeric product to be produced from the chemical composition;
receiving, via a user interface, one or more initial chemical components for inclusion in the recipe for preparing the chemical composition;
based on the at least one target physical property, determining, with at least one processor, whether inclusion of the one or more initial chemical components in the recipe would produce the polymeric product having the at least one target physical property;
generating, with at least one processor, an advisory component recommendation comprising one or more additional or alternative chemical components for inclusion in the recipe;
in response to a user accepting the advisory component recommendation, modifying the recipe to include the one or more additional or alternative chemical components; and
preparing the chemical composition based on the modified recipe.

12. The method of claim 11, comprising using a programmed or configured composition preparation device to prepare the chemical composition based on the modified recipe.

13-17. (canceled)

18. A system for creating a recipe for a chemical composition comprising at least one processor programmed or configured to:
receive, via a user interface, a property input from a user, the property input comprising at least one target physical property for a polymeric product to be produced from the chemical composition;
receive, via a user interface, one or more initial chemical components for inclusion in the recipe for preparing the chemical composition;
based on the at least one target physical property, determine whether inclusion of the one or more initial chemical components in the recipe would produce the polymeric product having the at least one target physical property;
generate an advisory component recommendation comprising one or more additional or alternative chemical components for inclusion in the recipe; and
in response to a user accepting the advisory component recommendation, modify the recipe to include the one or more additional or alternative chemical components.

19. The system of claim 18, wherein the at least one processor is further programmed or configured to:
in response to a user rejecting the advisory component recommendation, receive, via a user interface, one or more alternative or additional subsequent chemical components for inclusion in the recipe;
based on the at least one target physical property, determine whether inclusion of the one or more subsequent alternative or additional chemical components in the recipe would produce the polymeric product having the at least one target physical property;
generate an updated advisory component recommendation comprising one or more additional or alternative chemical components;
in response to a user accepting the updated advisory component recommendation, modify the recipe to form an updated recipe for preparing the chemical composition to include the one or more additional or alternative chemical components.

20. The system of claim 18, further comprising a composition preparation device, wherein the at least one processor is further programmed or configured to:
program or configure the composition preparation device to prepare the chemical composition based on the modified recipe.

21. The system of claim 20, wherein the composition preparation device is configured to prepare the chemical composition based on the modified recipe.

22. The system of claim 20, wherein during preparation of the chemical composition, the composition preparation device is configured to communicate feedback data to the at least one processor, the feedback data associated with a physical property of the chemical composition being prepared; and
the at least one processor is configured to generate an adjustment component recommendation based on the feedback data.

23. The system of claim 18, wherein the at least one processor is further programmed or configured to:
store the modified recipe in a saved recipes database.
